(19) United States
(12) Patent Application Publication
(10) Pub. No.: US 2023/0204483 A1
(43) Pub. Date: Jun. 29, 2023

(54) YOGHURT CREAMINESS

(71) Applicant: DSM IP ASSETS B.V., Heerlen (NL)

(72) Inventors: Cornelia Elizabeth Paulina MALJAARS, Echt (NL); Margriet Megtilda Wilhelmina Bartholomea HENDRIKS, Echt (NL); Filip Stefan Emiel OOSTERLINCK, Echt (NL)

(21) Appl. No.: 17/999,571

(22) PCT Filed: May 20, 2021
(86) PCT No.: PCT/EP2021/063563

§ 371 Date: Nov. 22, 2022

(30) Foreign Application Priority Data
May 25, 2020 (EP) 20176334.9

(51) Int. Cl. G01N 11/00; A23C 9/123; G01N 11/04; G01N 11/10

(52) U.S. Cl. CPC G01N 11/00; A23C 9/1238; G01N 11/04; G01N 11/10

(57) ABSTRACT

The present invention relates to a method for selecting a yoghurt sample with a creamy mouthfeel, comprising the steps of:
(i) providing one or more yoghurt samples;
(ii) determining from the one or more yoghurt samples the time to breakup using an extensional rheometer; and
(iii) optionally, measuring the viscosity and/or shear stress of the one or more yoghurt samples; and
(iv) selecting one or more yoghurt samples.

### FIELD OF THE INVENTION

The present invention relates to a method for selecting a yoghurt sample having a creamy mouthfeel. Further, the present invention relates to a method for ranking of microorganisms according to their ability to contribute to creamy mouthfeel. Further, the present invention relates to a method for manufacturing a starter culture composition comprising mixing lactic acid bacteria.

### BACKGROUND

The food industry uses different bacteria, in the form in particular of ferments, in particular lactic acid bacteria, in order to improve the taste and the texture of foods but also to extend the shelf life of these foods. In the case of the dairy industry, lactic acid bacteria are used intensively in order to bring about the acidification of milk (by fermentation) but also in order to texturize the product into which they are incorporated. Among the lactic acid bacteria used in the food industry, there can be mentioned the genera Streptococcus and Lactobacillus. The lactic acid bacterial species Streptococcus thermophilus and Lactobacillus delbrueckii ssp bulgaricus are used in particular in the formulation of the ferments used for the production of fermented milks, for example yoghurts.

The acidity produced in yoghurt depends mainly on the acidifying activity of the yoghurt culture (Streptococcus thermophilus and Lactobacillus delbrueckii ssp. bulgaricus) and therefore the amount of lactic acid produced during the milk maturation and also the residual acidity produced during cold storage. The texture also contributes to the final product sensorial properties. The recipe of the yoghurt has also an impact on the yoghurt sensorial properties by affecting the texture or the aroma perception.

The characteristics of yoghurt are commonly assessed in series of sensory studies. In sensory studies, so called sensory attributes are evaluated by a sensory panel, for example following the Quantitative Descriptive Analysis (QDA) method. The Quantitative Descriptive Analysis (QDA) method was developed by Tragon Corporation in 1974 and is a behavioral sensory evaluation approach that uses descriptive panels to measure a product's sensory attributes. It is described in detail in many handbooks, for example by Stone et al. in chapter 1.3, titled “ Sensory Evaluation by Quantitative Descriptive Analysis”, in the handbook titled “Descriptive Sensory Analysis in Practice”, edited by by Gacula Jr, published by Food & Nutrition Press Inc., USA, in 2004. Sensory attributes can be divided as attributes relating to structure (thickness, ropiness, smoothness, shininess and odour), sensory attributes related to mouthfeel (firmness, viscosity, graininess, slipperiness, creamy mouthfeel, sliminess and melting), and attributes relating to flavour (intensity, sour, milk/cream, green, sweet, bitter and off-flavour). Attributes relating to structure are usually assessed by a spoon. Attributes relating to mouthfeel and flavour are usually assessed orally.

Sensory studies are very time consuming. Assessing information about yoghurt samples by means of instrumental measurements would therefore be very useful.

An important quality indicator for yoghurt is the creamy mouthfeel. Creamy mouthfeel is perceived as pleasant and indulgent by consumers. Therefore, it is important to be able to target product development of products with high creamy mouthfeel. As a result, it is desired to identify strains and/or strain combinations that provide products with high creamy mouthfeel. Currently, creamy mouthfeel is assessed best by sensory studies. During oral processing a complex series of manipulations is involved by which both physical and chemical properties of the yoghurt change owing to interactions with saliva and oral mucosa. Creamy mouthfeel depends on properties originating from both the bulk and the surface of the yoghurt. Creamy mouthfeel is a complex sensory attribute that reported to correlate multiple sensory properties, including smoothness, thickness, and specific flavours.

Dickinson et al ‘on the road to understanding and control creaminess perception in food colloids’, Food Hydrocolloids 77, 2018, 372-385, reports that a rheological instrument for measuring creaminess could not easily be envisaged, and suggests the additional importance of particle size and lubrication/tribology. Therefore, there is a need in the art for instrumental measurements that correlate with creamy mouthfeel.

Jellema et al. ‘Relating the sensory sensation creamy mouthfeel in custards to rheological measurements’, J. Chemometrics, 2005; 19: 191-200 studies the relation between rheological parameters and creamy mouthfeel in custards. Sensory measurements are carried out using a sensory panel trained to the principles of QDA. Custards where rheologically characterized using a rheometer. Two types of small-deformation measurements were performed, via a dynamic (oscillatory) stress and frequency, and two types of large-deformation measurements, namely determination of a flow curve and steady shear rate measurement.

Surber et al. ‘Shear and extensional rheology of acid milk gel suspensions with varying ropiness’, J. of texture studies, 2020; 51:111-119 studies the structure attribute ropiness and reports that break up and extensional viscosity significantly increased with increasing ropiness.

U.S. Pat. No. 4,965,079A relates to another technical field than yoghurts and seeks to provide an acidified milk product of creamy consistency which may contain little, if any, fats, which does not coagulate either during sterilization or when poured, for example, into hot coffee. U.S. Pat. No. 4,965,079A describes that it came as a surprise that certain roping “Lactobacillus bulgaricus” and the thickening “Streptococcus”, strains were capable of acidifying a milk suspension, while, at the same time, producing roping and thickening agents sufficiently to provide the milk suspension with a creamy consistency. The roping “Lactobacillus bulgaricus” is said to seemingly produce an agent having a roping effect. The thickening “Streptococcus” is said to seemingly produce an agent having a thickening effect. There was no particular method applied to assess creamy mouthfeel or to select the strains. To the contrary. The found effect is described as being a surprise.

U.S. Pat. No. 4,965,079A therefore also stresses the desirability in the art for instrumental measurements that correlate with creamy mouthfeel, allowing the person skilled in the art to predict creamy mouthfeel rather than having to apply trial and error.

In the article by Priyashantha: “Type of starter culture influences on structural and sensorial properties of low protein fermented gels”, J. of Texture Studies, Vol. 50(6), (2019), pages 482-492, a study is described that had the objective to understand how the choice of starter culture influence the gel sensorial and structural properties and to assess the correlations between instrumental and sensory properties of low protein fermented gels. The intensity of creaminess of the stirred gel was measured as a stringency/roughness (Baja áspero), more creamy/softness (Alta suave) at the mouth. The desirability in the art for instrumental measurements that correlate with creamy mouthfeel hence still existed in 2019.

WO2015/086574 states that texture can be described either by sensory analysis (performed by a panel of trained people) or by rheological methods providing information about flow behavior and viscous/elastic characters of the product. It is stated that creaminess is often associated with high ropiness, but does not provide any further detail.

Creamy mouthfeel is a complex sensory attribute that reported to correlate multiple sensory properties, including smoothness, thickness, and specific flavours. WO2015/086574 does not provide any teaching if or how instrumental measurements could be used to predict such creamy mouthfeel. Viscosity, as for example determined by Brookfield viscosity or shear rheology measurements, relates to the mouthfeel attribute creamy-mouthfeel and is therefore used to identify strains/strain combinations that have the potential to provide creamy mouthfeel in fermented milk products. However, as this is not a 1 to 1 relationship, this regularly leads to false positives (products with high viscosity that fail to provide the expected high creamy mouthfeel) or false negatives (products with medium viscosity that have a higher creamy mouthfeel than expected based on viscosity only). There is a need in the art for improved methods to identify yoghurts having a creamy mouthfeel and to identify lactic acid bacteria strains that are able to provide yoghurt having a creamy mouthfeel.

### SUMMARY OF THE INVENTION

The inventors have now surprisingly found a new method that provides an improved ranking of strains/strain combinations on creamy mouthfeel, by combining shear rheology (viscosity) and extensional rheology.

In a first aspect the present invention relates to a method for selecting a yoghurt sample with a creamy mouthfeel, comprising the steps of:
(i) providing one or more yoghurt samples;
(ii) determining from the one or more yoghurt samples the time to breakup using an extensional rheometer; and
(iii) optionally, measuring the viscosity and/or shear stress of the one or more yoghurt samples; and
(iv) selecting one or more yoghurt samples.
   The present inventors found that the time to breakup of a yoghurt sample correlates to the creamy mouthfeel of the yoghurt sample as perceived by the consumer. Hence, the time to breakup can be advantageously used for identifying yoghurts having a creamy mouthfeel. This is surprising in that creamy mouthfeel is a complex sensory attribute for which no good correlation with instrumental measurements was found yet.

The present inventors have further found that additional efficiency in identifying yoghurts having a creamy mouthfeel can be obtained by combining a measurement of the time to breakup of one or more yoghurt samples using an extensional rheometer and a measurement of the viscosity and/or shear stress of the one or more yoghurt samples. For example, when a collection of yoghurt samples is provided, it is advantageous to measure the time to breakup of each yoghurt sample and measure the viscosity and/or shear stress of each yoghurt sample and subsequently select only those yoghurt samples having both a higher than average breakup time and a higher than average viscosity, respectively a higher than average shear stress, and subsequently subject only the selected yoghurt samples to a sensory study. By having only these yoghurt samples tested by the sensory panel, valuable efficiency in time and cost can be gained.

With the above method also additional time and cost efficiency can be gained when testing and selecting new micro-organisms, such as new lactic acid bacteria.

In a second aspect the present invention therefore provides a method for ranking of microorganisms according to their ability to contribute to creamy mouthfeel, comprising the steps of:
(1) providing yoghurt samples;
(2) determining from two or more yoghurt samples the time to breakup using an extensional rheometer;
(3) optionally, measuring the viscosity and/or shear stress of the one or more yoghurt samples; and
(4) ranking the microorganisms according to their ability to contribute to creamy mouthfeel by ranking the yoghurt samples on time to breakup.

In a third aspect, the present invention provides a method for manufacturing a starter culture composition comprising mixing lactic acid bacteria, wherein one or more of the lactic acid bacteria are identified by ranking the lactic acid bacteria according to their ability to contribute to creamy mouthfeel as described above or selecting a yoghurt sample as described above.

### DETAILED DESCRIPTION

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

The various embodiments of the invention described herein may be cross-combined.

The term “creamy mouthfeel”, or creaminess mouthfeel, is defined as the degree to which the product gives a full and creamy mouthfeel wherein the product does not feel rough, it is not dry and it has a velvety coating. Creamy mouthfeel is preferably evaluated by putting a spoonful of yoghurt in the mouth, followed by manipulating the product by making hewing/rolling movements against the palate. Trained sensory panellists can preferably rate the perceived creamy mouthfeel on an unstructured line scale, wherein the lower end of the unstructured line scale is defined as very little creamy mouthfeel, whereas the higher end is defined as very much creamy mouthfeel. Creamy mouthfeel can suitably be analysed via the quantitative descriptive analysis (QDA) as illustrated by the examples.

As used herein, the term “yoghurt” is a fermented milk product produced by fermentation of milk by lactic acid bacteria, also known as “yoghurt cultures”.

Preferably the milk is a mammal milk. The fermentation of the lactose in milk produces lactic acid which acts on the milk protein to give the yoghurt its texture. Yoghurt may be made from cow milk, the protein of which mainly comprises casein, which is most commonly used to make yoghurt, but milk from sheep, goat, buffalo, camel, llama, mare, deer, water buffalo, ewes and/or mares, and combinations thereof may be used as well. The term “yoghurt” furthermore encompasses, but is not limited to, yoghurt as defined according to French and European regulations, e.g. coagulated dairy products obtained by lactic acid fermentation by means of specific thermophilic lactic acid bacteria only (i.e. Lactobacillus delbruekii subsp. bulgaricus and Streptococcus thermophilus) which are cultured simultaneously and are found to be living in the final product in an amount of at least 10 million CFU (colony-forming unit) per gram of the yoghurt. Preferably, the yoghurt is heat-treated after fermentation or is not heat-treated after fermentation. Yoghurts may optionally contain added dairy raw materials (e.g. cream and/or protein) or other ingredients such as sugar or sweetening agents, one or more flavouring(s), cereals or nutritional substances, especially vitamins, minerals, stabilizers and fibers. Such yoghurt advantageously meets the specifications for fermented milks and yoghurts of the AFNOR NF 04-600 standard and/or the codex StanA-IIa-1975 standard. In order to satisfy the AFNOR NF 04-600 standard, the product must not have been heated after fermentation and the dairy raw materials must represent a minimum of 70 wt % of the finished product.

Yoghurt encompasses set yoghurt, stirred yoghurt, drinking yoghurt, Petit Suisse, heat treated yoghurt and yoghurt-like products. Preferably, the yoghurt is a stirred yoghurt, a set yoghurt or a drinking yoghurt. More preferably, the yoghurt is a stirred yoghurt or a set yoghurt.

The term “yoghurt” further comprises yoghurt analogues such as plant based yoghurts. Such plant based yoghurts can be prepared from plant based milks such as for example soy milk, coconut milk, or almond milk. Optionally one or more sugars can be added to such plant based milk.

Preferably the yoghurt is a yoghurt based on mammal milk, as described above. Such yoghurt based on a mammal milk can also be referred to as “mammal yoghurt” or “non-plant based yoghurt”.

The term “providing one or more yoghurt samples” as used in the present context means either the provision of a single yoghurt sample or the provision of a multitude of yoghurt samples.

Preferably, present step (i), respectively step (1), of providing one or more yoghurt samples comprises providing of a collection of yogurt samples. More preferably a collection of yoghurt samples wherein yoghurt samples are produced by different lactic acid bacteria or by different combinations of lactic acid bacteria. Alternatively, a collection of yoghurt samples wherein yoghurt samples are produced by using different milk recipes. A collection of yoghurt samples comprises preferably equal to or more than 20 yoghurt samples, more preferably equal to or more than 50 yoghurt samples, more preferably equal to or more than 100 yoghurt samples, more preferably equal to or more than 150 yoghurt samples.

As indicated above, the yoghurt samples are preferably set or stirred yoghurt samples.

In a preferred embodiment, the present method further comprises measuring the viscosity and/or shear stress of the one or more yoghurt samples. Alternatively, present step (i) of providing one or more yoghurt samples comprises providing one or more yoghurt samples that are selected using measuring their viscosity and/or shear stress. As explained above, the present inventors found that breakup time improves the correlation of viscosity and /or shear stress with creamy mouthfeel, and thus it is beneficial to combine viscosity and/or shear stress with measuring the breakup time.

Preferably, the present method comprises the step of determining from one or more yoghurt samples the viscosity and/or the shear stress. More preferably this step is carried out before, after, or simultaneously with present step (ii), respectively step (2), of the methods described above. Suitably such a step can optionally be present as a step (iii), respectively a step (3) in the methods described above.

Preferably the viscosity of the one or more yoghurt samples is measured by a Brookfield viscometer and/or the shear stress of the one or more yoghurt samples is measured by shear rheology.

Most preferably the method according to a first aspect of the invention therefore comprises a method for selecting a yoghurt sample with a creamy mouthfeel, comprising the steps of:
(i) providing a collection of yoghurt samples;
(ii) determining from each of the yoghurt samples the time to breakup using an extensional rheometer; and
(iii) optionally, measuring the viscosity and/or shear stress of each of the yoghurt samples; and
(iv) selecting one or more yoghurt samples from the collection of yoghurt samples.

The method according to the first aspect of the invention can be used as an alternative to an evaluation by a sensory panel, thus completely excluding the use of such a sensory panel. In this case the method is carried out in the absence of an evaluation by a sensory panel. This allows for the advantageous avoidance of all the costs and time involved in such an evaluation.

However, the method according to the first aspect of the invention can also be advantageous if combined with an evaluation by a sensory panel. By applying the method according to the invention to a collection of yoghurt samples, the number of yoghurt samples to be tested in an evaluation by a sensory panel can be greatly reduced. Such a reduction in samples advantageously saves time and costs and increases efficiency.

There is therefore also provided a preferred method for selecting a yoghurt sample with a creamy mouthfeel, comprising the steps of:
(i) providing a collection of yoghurt samples;
(ii) determining from each of the yoghurt samples the time to breakup using an extensional rheometer; and
(iii) optionally, measuring the viscosity and/or shear stress of each of the yoghurt samples; and
(iv) selecting one or more yoghurt samples from the collection of yoghurt samples; and
(v) optionally, submitting the selected yoghurt samples to an evaluation by a sensory panel.

The sensory panel can be a human sensory panel, but can also be an artificial sensory panel, i.e. a sensory panel based on artificial intelligence. Preferably the sensory panel is a human sensory panel.

The term “the time to breakup” as used in the present context is defined as the time for a fluid column to thin from a minimum diameter of 1.5 mm to a minimum diameter smaller than 5 μm using an extensional rheometer. Preferably, the present extensional rheometer is a capillary breakup extensional rheometer, also referred to simply as “capillary extensional rheometer”. Capillary extensional rheometers are configured to quantify the elongational properties of a fluid or semi fluid like yoghurt.

The term “selecting one or more yoghurt samples” as used in the present context means selecting one or more yoghurt samples from a collection of yoghurt samples or from a single yoghurt sample.

In a preferred embodiment, creamy mouthfeel is evaluated by: descriptive testing of the attribute creamy mouthfeel according the Quantitative Descriptive Analysis (QDA). To this end, a highly trained Fermented Milk Products panel assessed the products taking into account Good Sensory Practices. During the QDA measurements, the intensities of the attribute was obtained by EyeQuestion, using unstructured line scales ranging from 0-100. The yoghurt samples were evaluated in duplicate according to a balanced incomplete block design. The yoghurt samples were stored in the refrigerator at 5° C. The yoghurt samples were given to the panellists one-by-one, in a white polystyrene cup with a white polystyrene spoon to assess the yoghurt sample. Creamy mouthfeel is evaluated by putting a spoonful of yoghurt in the mouth, followed by manipulating the product by making hewing/rolling movements against the palate. The lower end of the unstructured line scale is defined as very little creaminess mouthfeel, whereas the higher end is defined as very much creaminess mouthfeel.

In a preferred embodiment, the present selecting one or more yoghurt samples comprises selecting one or more yoghurt samples having a higher time to breakup. More preferably a higher time to breakup than different yoghurt samples or a higher time to breakup than the average time to break up of the yoghurt samples.

Preferably, the present step (iv), respectively step (4) of selecting one or more yoghurt samples comprises selecting one or more yoghurt samples based the time to breakup as determined in present step (ii), respectively step (2). More preferably, the present step (iv), respectively step (4), of selecting one or more yoghurt samples comprises selecting one or more yoghurt samples based a longer time to breakup as determined in present step (ii), respectively step (2) as compared to other yoghurt samples. The time to breakup can also be saved or collected in a database providing the advantage that new determined times to breakup can be easily compared with the database to provide the comparative data. In this example single yoghurt samples can be provided, determined and selected using the method of the present invention.

In a preferred embodiment, present step (iv), respectively step (4) of selecting one or more yoghurt samples comprises selecting one or more yoghurt samples having a creamy mouthfeel. Given the found correlation between time to break up as determined in present step (ii), respectively step (2) and creamy mouthfeel the present invention allows directly for selecting yoghurt samples with a creamy mouthfeel.

In addition, step (iv), respectively step (4) may comprise selecting one or more yoghurt samples having a higher Brookfield viscosity and/or a higher shear stress, for example as determined in optional step (iii), respectively optional step (3).

Suitably the higher values can be determined by calculating the average of all the values as determined for a particular parameter, for example as determined for the breakup time, Brookfield viscosity and/or shear stress, within a certain collection of yoghurt samples, and then selecting the values which are above such average. More preferably those values which are within equal to or less than the highest 40%, equal to or less than the highest 30%, equal to or less than the highest 20%, equal to or less than the highest 10% or equal to or less than the highest 5% of the values a particular parameter, for example as determined for the breakup time (i.e. the time for breakup), Brookfield viscosity and/or shear stress, are determined. The yoghurt samples for which such highest values were achieved can subsequently be selected.

There is therefore also provided a preferred method for selecting a yoghurt sample with a creamy mouthfeel, comprising the steps of:
(i) providing a collection of yoghurt samples;
(ii) determining for each of the yoghurt samples the time to breakup using an extensional rheometer and determining for which yoghurt samples the value for such time to breakup is above the average, or within equal to or less than the highest 40%, or within equal to or less than the highest 30%, or within equal to or less than the highest 20%, or within equal to or less than the highest 10%, or within equal to or less than the highest 5%, of the collection; and
(iii) optionally, measuring the viscosity with a Brookfield viscometer and/or shear stress by shear rheology of each of the yoghurt samples and determining for which yoghurt samples the value for such viscosity and/or shear stress is above the average, or within equal to or less than the highest 40%, or within equal to or less than the highest 30%, or within equal to or less than the highest 20%, or within equal to or less than the highest 10%, or within equal to or less than the highest 5%, of the collection; and
(iv) selecting the one or more yoghurt samples from the collection of yoghurt samples for which:
   the value for the time to breakup is above the average, or within equal to or less than the highest 40%, or within equal to or less than the highest 30%, or within equal to or less than the highest 20%, or within equal to or less than the highest 10%, or within equal to or less than the highest 5%, of the collection; and
   optionally the value for the viscosity and/or shear stress is above the average, or within equal to or less than the highest 40%, or within equal to or less than the highest 30%, or within equal to or less than the highest 20%, or within equal to or less than the highest 10%, or within equal to or less than the highest 5%, of the collection; and
(v) optionally, submitting the selected yoghurt samples to an evaluation by a sensory panel.

In a preferred embodiment, the present one or more yoghurt samples have a volume of less than 1 ml, more preferably less than 0.5 ml, even more preferably less than 0.2 ml, or more preferably less than 0.1 or less than 0,05 ml. The advantage of using an extensional rheometer is that small samples volumes can be used. This is beneficial in that less yoghurt is needed for a reliable determination of the creamy mouthfeel. Hence, by following the method of the present invention, a more efficient method for selecting a yoghurt sample with creamy mouthfeel is provided. Further, using small sample volumes allows a high throughput workflow for selecting yoghurt samples with creamy mouthfeel.

The above described methods for selecting a yoghurt sample can advantageously be used for the selection of one or more micro-organisms that have an ability to contribute to creamy mouthfeel.

Hence, according to another aspect, the present invention relates to a method for ranking of microorganisms according to their ability to contribute to creamy mouthfeel, comprising the steps of:
(1) providing yoghurt samples;
(2) determining from two or more yoghurt samples the time to breakup using an extensional rheometer;
(3) optionally, measuring the viscosity and/or shear stress of the one or more yoghurt samples; and
(4) ranking the microorganisms according to their ability to contribute to creamy mouthfeel by ranking the yoghurt samples on time to breakup.

The identified correlation between time to breakup in an extensional rheometer and creamy mouthfeel of a yoghurt sample allows the identification of microorganisms according to their ability to contribute to creamy mouthfeel.

The yoghurt samples in step (1) can suitably be prepared by fermenting a milk whilst using one or more microorganisms. Fermentation of a milk to prepare a yoghurt is well known and for example described in the handbook by Tamine and Robinson, titled “Yoghurt Science and Technology”, published by Woodhead Publishing Limited, (2000), paragraph 1.5. A suitable example of such a preparation process is exemplified in the examples.

Unless explicitly stated otherwise, preferences as described above for steps (i), (ii) and (iii) apply mutatis mutandis to corresponding steps (1), (2) and (3).

The preferences as described above for step (iv) can advantageously be applied to rank the yoghurt samples in step (4) and consequently the microorganisms that were used to prepare such yoghurt samples.

In a preferred embodiment, the microorganisms are lactic acid bacteria. More preferably the present microorganisms are Streptococcus thermophilus and/or Lactobacillus delbruekii subsp. Bulgaricus.

As used herein, the term “lactic acid bacteria” (LAB) or “lactic bacteria” refers to food-grade bacteria producing lactic acid as the major metabolic end-product of carbohydrate fermentation. These bacteria are related by their common metabolic and physiological characteristics and are usually Gram positive, low-GC, acid tolerant, non-sporulating, non-respiring, rod-shaped bacilli or cocci. During the fermentation stage, the consumption of lactose by these bacteria causes the formation of lactic acid, reduces the pH and leads to the formation of a (milk) protein coagulum. These bacteria are thus responsible for the acidification of milk and for the texture of the fermented milk product. As used herein, the term “lactic acid bacteria” or “lactic bacteria” encompasses, but is not limited to, bacteria belonging to the genus of Lactobacillus spp., Bifidobacterium spp., Streptococcus spp., Lactococcus spp., such as Lactobacillus delbruekii subsp. bulgaricus, Streptococcus thermophilus, Lactobacillus lactis, Bifidobacterium animalis, Lactococcus lactis, Lactobacillus casei, Lactobacillus plantarum, Lactobacillus helveticus, Lactobacillus acidophilus and Bifidobacterium breve.

In a preferred embodiment, the present method further comprises step (iv) of selecting at least one microorganism if it contributes to a creamy mouthfeel. Whether or not a microorganism contributes to a creamy mouthfeel can be identified using the determination of the breakup time using an extensional rheometer. Yoghurt samples prepared with different combinations of lactic acid bacteria or with different starter culture compositions can be analysed using the extensional rheometer. By comparing the different breakup times per different combination of lactic acid bacteria, the present invention allows to select microorganisms that contribute to a creamy mouthfeel.

According to another aspect, the present invention relates to a method for manufacturing a starter culture composition comprising mixing lactic acid bacteria, wherein one or more of the lactic acid bacteria are identified by ranking the lactic acid bacteria according to their ability to contribute to creamy mouthfeel as defined above. Alternatively, the present invention relates to a method for manufacturing a starter culture composition comprising mixing lactic acid bacteria, further comprising the present method for selecting a yoghurt sample having a creamy mouthfeel and/or further comprising the present method for ranking of microorganisms according to their ability to contribute to creamy mouthfeel.

The term “starter culture composition” or “composition” (also referred to as “starter” or “starter culture”) as used herein refers to a composition comprising one or more lactic acid bacteria, which are responsible for the acidification of the milk base. Starter cultures compositions may be fresh (liquid), frozen or freeze-dried. Freeze dried cultures need to be regenerated before use. For the production of a fermented dairy product, the starter cultures composition is usually added in an amount from 0.01 to 3%, preferably from 0.01 and 0.02% by weight of the total amount of milk base.

### EXAMPLES

### Methods

### Yoghurt Preparation

Pasteurized semi-skimmed milk (Private label, Plus Supermarket, The Netherlands) was mixed with pasteurized full fat milk (Plus Supermarket, The Netherlands) and skimmed milk powder to obtain the recipes outlined in Table 1. The milk bases were homogenized in-line (80 over 40 bar) and pasteurized at 92° C. for 5 minutes. Subsequently, the pasteurized milk mix was inoculated with the cultures listed in the section “starter cultures” at an inoculation rate of 4 U/1000 L. The fermentation was performed at 42° C. in a water bath until a pH of 4.60 was reached. The yoghurts were smoothened using a back-pressure valve set at a back pressure of 1 bar on yoghurt and cooled to 22° C. before filling out in cups. In parallel, the pH was continuously recorded using a Cinac system.

TABLE 1
Overview of yoghurt recipes

| | 1 | 2 |
|---|---|---|
| Protein (%) | 4.2 | 4.2 |
| Fat (%) | 1.5 | 0 |

### Starter Cultures

Nine different starter cultures were used, including Delvo®Yog YS-242, FVV-221, MY-131, Biogarde 1, YG-441, FVV-231, FVV-511,FVV-111, FVV-122, all commercially available from DSM Food Specialties, Delft, The Netherlands. The cultures were analyzed in a blind/randomized manner and labelled Starter culture A-I.

### Measurement of Time to Breakup

Capillary break up (CABER) measurements were used to determine the extensional rheology of yogurt cultures. With the CABER method a thin filament is created and the evolution of the diameter is followed in function of time (the surface tension drives to break up the filament and needs to overcome the resistance offered by the extensional rheology). The break-up time of the rope (i.e. how long the thinning filament) is taken as a quantitative value for the extensional rheology of the yoghurts. The measurement itself was performed using a self-build setup, comparable to the commercial available HAAKE™ CaBER™ Capillary Breakup Extensional Rheometer. The set-up consist of a linear motor, capable of performing a step-change with controlled speed over a pre-defined distance at a speed of 60 mm/s. To the linear motor, a circular plateau with a diameter of 4 mm is attached, plan parallel to a second, level plateau with a diameter of 4 mm. Perpendicular to the linear motor, an IDT NR4-3S CMOS camera is mounted, capable of recording 1024×1024 pixels at 3000 fps and provided with a Edmund Optics 0.7×-4.5× coaxial zoom lens. The camera is attached to an xy-table for proper alignment and focusing.

To facilitate a homogeneous sample while minimizing the risk of damaging the texture, a 125 ml cup containing the sample was tilted to 60° angle with the lid facing upwards, and rotated 5 times around its longitudinal axis. This was repeated with the lid facing downwards.

At the start of the measurement, a gap height of 2 mm is used between the circular plateaus. A 0.025 ml sample was brought into this gap to a level that small bulging of the sample fluid is visible, using a syringe with a blunted needle (18G). The experiment starts by a quick (50 ms) step-change movement from an initial gap height of 2 mm to a gap height of 5.5 mm. This step-change will stretch the fluid sample, ultimately pinching off the formed fluid column/filament. The experiment, starting from the step-change and ending by the breaking of the stretched fluid column is recorded by the IDT NR4-3S camera at a 4.5× magnification. Back-lighting, by IDT-LED lighting is used for proper view (contrast) of the fluid column.

The recordings are evaluated by determining the minimum diameter of the fluid column over time, using a Sobel edge detection algorithm. The FOV (field of view) of the camera is calibrated using a microscope object glass with 1/100 scaling (WILD Heerbrugg Switserland calibration slide). The break-up time is now defined by the time for a fluid column to thin from a minimum diameter of 1.5 mm to a minimum diameter smaller than 5 μm (minimum detection level of the Sobel edge detection with the used magnification and camera set-up) and is usually expressed in milliseconds (ms).

### Measurement of Shear Stress

The samples were measured using a Physica MCR302 rheometer equipped with a concentric cylinder measurement system (CC-27). Yoghurt samples were stored at 4° C. and were taken out of storage just prior to measuring in the rheometer, with the containers having to be handled with extreme care. To facilitate a homogeneous sample while minimizing the risk of damaging the texture, a 125 ml cup containing the sample was tilted to 60° angle with the lid facing upwards, and rotated 5 times around its longitudinal axis. This was repeated with the lid facing downwards.

Once filled, the measuring cup was placed into the rheometer and superfluous material was removed by a pipette. The procedure to load the yoghurts took about two minutes. Care needs to be taken to treat all different samples in exactly the same way, since difference in loading conditions can cause differences in the relative ranking of the yoghurts. Before measurement the samples were allowed to rest and equilibrate to the measuring temperature (25° C.) for 5 minutes.

A standard experimental protocol was applied consisting out of the following two measuring sequences:
1. A strain sweep to determine the initial gel strength (dynamic shear modulus): an oscillatory test where at a fixed angular frequency (omega=10 rad/s) an increasing amplitude was applied: on a logarithmic scale the amplitude was increased from 0.01 to 100% with 5 measuring points per decade.
2. After the strain sweep the yoghurts were allowed to rest for 30 seconds in the rheometer and subsequently a shear rate sweep was applied to determine the shear stress in mouth: This consisted of applying an increasing shear rate to the yoghurts ranging from 0.001 to 1000 s-1 on a logarithmic scale with 3 measuring points per decade (no fixed time setting: the rheometer software determined the required shearing time per measuring point).

This experiment gave a flow curve whereby the measured stress was plotted as a function of the applied shear rate. For the perceived shear stress in the mouth, the shear stress at a flow rate of around 200 s⁻¹ can be used. Since the measured flow curve measures a point at 215 s⁻¹, the shear stress at that point is taken as a representative value for perceived shear stress in the mouth.

### Viscosity Measurement

Viscosity measurements were performed using a Brookfield RVDVII+ Viscometer, which allows viscosity measurement on an undisturbed product (directly in the pot). The Brookfield Viscometer determines viscosity by measuring the force required to turn the spindle into the product at a given rate. The Helipath system with a T-B spindle was used as it is designed for non-flowing thixotropic material (gels, cream). It slowly lowers or raises a rotating T-bar spindle into the sample so that not always the same region of the sample is sheared (helical path). Thus, the viscometer measures constantly the viscosity in fresh material, and is thus thought to be the most suitable for measuring stirred yoghurt viscosity. For the measurements, the spindle was placed on the surface of a cup containing approximately 125 ml yoghurt. The spindle was rotated at a speed of 30 rpm and 31 measuring points were recorded, at an interval of 3 sec. The average of the values between 60 and 90 seconds is reported. All samples were measured in triplicate.

### Measurement of Creamy Mouthfeel

Descriptive testing was performed according the Quantitative Descriptive Analysis (QDA) method to assess the products on their structure, mouthfeel, odor, flavor, after taste and after feel performance. To this end, a highly trained Fermented Milk Products panel assessed the products taking into account Good Sensory Practices. During the QDA measurements, the intensities of the attributes were obtained by EyeQuestion, using unstructured line scales ranging from 0-100. The samples were evaluated in duplicate according to a balanced incomplete block design. The products were stored in the refrigerator at 5° C. The products were given to the panellists one-by-one, in a white polystyrene cup with a white polystyrene spoon to assess the yoghurt. In total, 24 attributes were evaluated, related to structure (thickness, ropiness, smoothness, shininess), odour (intensity, sour), mouthfeel (firmness, viscosity, graininess, slipperiness, creaminess, sliminess, melting), flavor (intensity, sour, milk/cream, green, sweet, bitter, off-flavour), aftertaste (intensity, length) and afterfeel (astringency, mouthcoating).

Creamy mouthfeel is evaluated by putting a spoonful of yoghurt in the mouth, followed by manipulating the product by making hewing/rolling movements against the palate. The lower end of the unstructured line scale is defined as very little creaminess mouthfeel, whereas the higher end is defined as very much creaminess mouthfeel.

### Example 1

Selection of a starter culture with the most creamy mouthfeel based on Brookfield viscosity only, versus a combination of Brookfield viscosity and extensional rheology.

Yoghurt was made according to the method described in the Materials and Methods with starter cultures A-J as indicated above and the recipes 1 and 2 as defined in Table 1.

Subsequently, on day 7 after fermentation, all yoghurts were subjected to a sensory analysis including the attributes as described in Materials and Methods. In addition, the Brookfield viscosity, the break-up time by extensional rheology and the shear stress by shear rheology of the yoghurts was measured on day 7 after fermentation.

The results of the sensory analysis and Brookfield viscosity measurements are shown in Table 2 below. The results in Table 2 indicate that both in recipe 1 and 2, the yoghurt with the highest Brookfield viscosity value does not have the highest creamy mouthfeel. In contrast, as also illustrated by Table 2, the yoghurt with a high Brookfield viscosity, combined with the highest extensional rheology value (i.e. highest break-up time) leads to the selection of the yoghurt with the highest creamy mouthfeel. Therefore, it can be concluded that the combination of Brookfield viscosity and extensional rheology is superior to Brookfield viscosity only when selecting yoghurts on creamy mouthfeel.

Table 3 shows the results of the sensory analysis, shear stress as determined by shear rheology measurements and break-up time as determined by extensional rheology measurements of Yoghurts prepared with starter cultures A-I in recipes 1 and 2.

TABLE 2
Results of the sensory analysis, Brookfield viscosity measurements and
extensional rheology measurements of Yoghurts prepared with starter
cultures A - I in recipes 1 and 2.

| | | | Average of | Average of |
|---|---|---|---|---|
| | | Creamy | Brookfield viscosity | Breakup time |
| Culture | Recipe | mouthfeel | (mPa · s) | (in ms) |
| D | 1 | 52.7 | 6206.4 | 0.046 |
| H | 1 | 50.6 | 6453.3 | 0.042 |
| F | 1 | 48.9 | 6429.5 | 0.030 |
| C | 1 | 44.2 | 5877.2 | 0.049 |
| E | 1 | 41.9 | 5605.3 | 0.022 |
| A | 1 | 41.5 | 5843.6 | 0.020 |
| C | 1 | 37.8 | 4770.9 | 0.013 |
| G | 1 | 35.7 | 5387.1 | 0.022 |
| I | 1 | 21.4 | 2640.0 | 0.004 |
| B | 1 | 21.4 | 2990.3 | 0.003 |
| C | 2 | 47.0 | 5008.9 | 0.069 |
| F | 2 | 46.2 | 5453.7 | 0.043 |
| A | 2 | 38.6 | 4783.0 | 0.036 |
| H | 2 | 36.1 | 4966.5 | 0.034 |
| G | 2 | 29.3 | 4426.7 | 0.022 |
| F | 2 | 29.3 | 5088.5 | 0.038 |
| D | 2 | 28.5 | 3912.7 | 0.015 |
| H | 2 | 27.5 | 4886.5 | 0.041 |
| E | 2 | 23.3 | 3545.5 | 0.016 |
| B | 2 | 18.4 | 2383.8 | 0.004 |
| I | 2 | 11.8 | 2090.1 | 0.004 |

TABLE 3
Results of the sensory analysis, Shear rheology measurements and
extensional rheology measurements of Yoghurts prepared with starter
cultures A - I in recipes 1 and 2.

| | | | Average of | Average of |
|---|---|---|---|---|
| | | Creamy | Shear stress | Breakup time |
| Culture | Recipe | mouthfeel | @ 215 s⁻¹ | (in ms) |
| D | 1 | 52.7 | 37.30236667 | 0.046 |
| H | 1 | 50.6 | 36.766 | 0.042 |
| F | 1 | 48.9 | 37.92873333 | 0.030 |
| C | 1 | 44.2 | 37.15053333 | 0.049 |
| E | 1 | 41.9 | 38.5758 | 0.022 |
| A | 1 | 41.5 | 34.7165 | 0.020 |
| C | 1 | 37.8 | 31.14973333 | 0.013 |
| G | 1 | 35.7 | 30.97226667 | 0.022 |
| I | 1 | 21.4 | 18.71926667 | 0.004 |
| B | 1 | 21.4 | 18.04896667 | 0.003 |
| C | 2 | 47.0 | 31.02173333 | 0.069 |
| F | 2 | 46.2 | 31.77513333 | 0.043 |
| A | 2 | 38.6 | 30.49333333 | 0.036 |
| H | 2 | 36.1 | 26.8804 | 0.034 |
| G | 2 | 29.3 | 25.62713333 | 0.022 |
| F | 2 | 29.3 | 29.6234 | 0.038 |
| D | 2 | 28.5 | 26.4004 | 0.015 |
| H | 2 | 27.5 | 29.97476667 | 0.041 |
| E | 2 | 23.3 | 26.86603333 | 0.016 |
| B | 2 | 18.4 | 15.94403333 | 0.004 |
| I | 2 | 11.8 | 12.03813333 | 0.004 |

1. A method for selecting a yoghurt sample with a creamy mouthfeel, comprising:
(i) providing one or more yoghurt samples;
(ii) determining from the one or more yoghurt samples the time to breakup using an extensional rheometer; and
(iii) optionally, measuring the viscosity and/or shear stress of the one or more yoghurt samples; and
(iv) selecting one or more yoghurt samples.

2. The method according to claim 1, wherein the yoghurt samples have a volume of less than 1 ml.

3. The method according to claim 1, wherein the yoghurt samples are set or stirred yoghurt samples.

4. The method according to claim 1, wherein the extensional rheometer is a capillary extensional rheometer.

5. The method according to claim 1, wherein in (iii) viscosity of the one or more yoghurt samples is measured by a Brookfield viscometer and/or the shear stress of the one or more yoghurt samples is measured by shear rheology.

6. The method according to claim 1, wherein (iv) selecting one or more yoghurt samples comprises selecting one or more yoghurt samples having a higher time to breakup.

7. The method according to claim 1, wherein (iv) selecting one or more yoghurt samples comprises selecting one or more yoghurt samples having a higher Brookfield viscosity and/or a higher shear stress.

8. The method according to claim 1, wherein (iv) selecting one or more yoghurt samples comprises selecting one or more yoghurt samples having a creamy mouthfeel.

9. A method for ranking of microorganisms according to ability to contribute to creamy mouthfeel, comprising:
(1) providing yoghurt samples;
(2) determining from two or more yoghurt samples a time to breakup using an extensional rheometer;
(3) optionally, measuring the viscosity and/or shear stress of the one or more yoghurt samples; and
(4) ranking the microorganisms according to ability to contribute to creamy mouthfeel by ranking the yoghurt samples on time to breakup.

10. The method according to claim 9, further comprising the (5) selecting at least one microorganism if it contributes to a creamy mouthfeel.

11. The method according to claim 9, comprising selecting at least one microorganism that contributed to a yoghurt sample having a higher time to breakup.

12. The method according to claim 9, comprising selecting at least one microorganism that contributed to a yoghurt sample having a higher Brookfield viscosity and/or a higher shear stress.

13. The method according to claim 9, wherein the microorganisms are lactic acid bacteria.

14. The method according to claim 9, wherein the microorganisms are Streptococcus thermophilus and/or Lactobacillus delbruekii subsp. Bulgaricus.

15. The method for manufacturing a starter culture composition comprising mixing lactic acid bacteria, wherein one or more of the lactic acid bacteria are identified by ranking the lactic acid bacteria according to their ability to contribute to creamy mouthfeel as determined in claim 9.
